(19) United States
(12) Patent Application Publication
(10) Pub. No.: US 2017/0035801 A1
(43) Pub. Date: Feb. 9, 2017

(54) ANTI-ITCHING AGENT

(71) Applicant: Lilac Laboratory Co., Ltd., Saitama-ken (JP)

(72) Inventor: Ryoichi HIRAIWA, Saitama-ken (JP)

(21) Appl. No.: 14/816,068

(22) Filed: Aug. 3, 2015

(51) Int. Cl. A61K 33/20; A61K 31/724

(52) U.S. Cl. CPC A61K 33/20; A61K 31/724

(57) ABSTRACT

The present invention relates to an anti-itching agent that prevents itch of skin. An emulsion, which is produced when sebum and sweat continuously secreted are mixed, contains a stimulatory composition having aqueous and oily properties and can be a causative agent of itch. In addition, the emulsion can be a cell disorder composition. Furthermore, the emulsion causes itching and becomes a causative agent triggering and promoting dermatitis. The problem can be solved by blocking the emulsion by a skin-degenerative stratum corneum, which is formed by an action of an aluminum chloride and water, the aluminum chloride and a cyclodextrin.

### BACKGROUND OF THE INVENTION

1. Field of the Invention

Since emulsion, which is a cell disorder composition produced when sebum and sweat (referred to as persistent sweat) continuously secreted during rest are mixed, is permeated into skin stratum corneum, spread and concentrated by body temperature, the emulsion is transmitted through the skin stratum corneum and easily reached to epidermal cells. Thus, sudden itch is caused. Furthermore, the emulsion of this state can be a causative agent of skin diseases such as eczema and dermatitis and can be a causative agent of itch. The present invention provides an anti-itching agent for preventing itch caused by the emulsion.

It is often considered that the concept that makes a person feel “itch” is common in all cases because the itch is felt in all cases. However, the itch can be categorized in the following eight variations.

(1) itch caused by sweat

(2) itch of follicle esthesioneurosis caused by static electricity

(3) itch caused by allergic reaction (urticaria, drug eruption, autosensitization dermatitis: atopy)

(4) itch accompanied with eczema and dermatitis caused by microorganism such as fungus and bacterium

(5) itch caused by diseases such as liver cirrhosis and dialysis patient

(6) toxinogenic itch caused by harmful insects (similar category as the allergic reaction)

(7) itch caused by drying (strictly, due to eczema and dermatitis)

(8) itch when removing eschar of external injury or surgical wound

The “itch” of the present invention is the itch caused by sweat categorized in the above described (1).

2. Description of Related Art

The dermatological theory up to the present sees that an emulsion composed of eccrine sweat, apocrine sweat, and sebum, which is secreted from the sebaceous gland, as a concept of moisturizing and protection effects. However, supposedly, even if water and the sebum have gone from the skin stratum corneum, as long as the epidermal cells themselves are not damaged, the skin is merely dried to the extent where the sulcus cutis looks white macroscopically. Functions such as stretchability of the epidermal cell layer are not lost at all. This is because that the stratum corneum serves as a barrier for the living epidermal cells.

Additionally, the generally spoken term, <dry skin> is extremely ambiguous. By appearance of eczema (dermatitis) of almost 100%, the originally living epidermal cells themselves are disordered, and extinct epidermal cells (scales), which lose their nuclei, are deposited. This state is merely referred to as the dry skin or xeroderma. As a result of extensive researches based on such knowledge, the inventor has found that this emulsion itself is a causative agent mainly causing the sudden itch. Thus, the inventor has reached the present invention.

Through skin practices for 19 years, a voice of patients from which I have realized is, regardless of age, a voice questioning “Although eruption gets better with medicine, is there anything that can be done about an itch?” A dogma by dermatologists/plastic surgery doctors is that treatment of curing the eruption and soothing the itch is rules and principles derived from medical treatment for health. The medical treatment is to cure diseases. With the stereotype, the medical treatment has been developed in the form of a progress of new medicines. However, in association with a recent expansion of medical cost, approaches to prevent diseases before getting sick have been gradually taken into consideration.

Most eczema and dermatitis in the field of dermatology increase during summertime, a warm period, and a period of having a warm in a winter season. Accordingly, ways of thinking: <getting itchy due to sweating> and <Sweating aggravates the eczema and the dermatitis.> are present. However, the main cause for such sudden itch has not been solved and also has not been problematized. As a result of affection with a disease where the cause of the itch is persistently the eczema and the dermatitis, it is regarded that the main reason for causing the itch is an isolation of chemical mediators, such as histamines, from leukocytes and irritating materials such as cytokines.

Meanwhile, the way of thinking that the sweating brings the itch has been a common opinion conventionally. It has been considered that in the concept of hidroschesis, inhibiting secretion of sweat and smoothing the skin inhibit miliaria, ensuring reducing the itch. Accordingly, baby powder has been widely used as commercial products. The main ingredients of the baby powder are starch such as corn starch, zinc oxide, talc, aluminum chlorohydrate, and a similar ingredient. The principle of the baby powder is as follows. Processed starch and silicic anhydride powder absorb the sweat. The zinc oxide and the talc sterilize the skin and improve the slipperiness. The antiperspirant effect by the aluminum chlorohydrate powder inhibits the sweating. When using the baby powder after toileting the sweat on the whole body, the touch is smooth and the person feels like that the itch has subsided to some extent. However, since the itch comes back soon, the baby powder has no effect to stop a scratch action. Dermatologists recommend the baby powder as an anti-itching agent and encourage many patients who have the itch to use the baby powder. However, since the use of the baby powder does not remove the itch under the actual situation, the majority opinion is that scratches are not cleared up.

### BRIEF SUMMARY OF THE INVENTION

The emulsion, which is produced especially when sebum and sweat continuously secreted during rest are mixed, contains a stimulatory composition having aqueous and oily properties. Here, the sweat includes acute sweat (persistent sweat). In addition, the emulsion is a cell disorder composition and a causative agent of itch. Since the emulsion is concentrated on the skin stratum corneum, the emulsion is transmitted through the stratum corneum and reached to epidermal cells causing itching. In addition, the emulsion becomes a causative agent of itch triggering and promoting the dermatitis.

The present invention has assumed as follows. The itch that cannot be solved by the antiperspirant effect is caused by the emulsion concentrated on the skin stratum corneum. The invention has focused on a water-repellent film effect from the aluminum chloride compound, inclusion and absorption effects from the cyclodextrin, and an interaction compounded with the water. Thus, the invention has solved the mechanism for itch.

Using the publicly-known and publicly used aluminum chloride compound as an antiperspirant, numerous sweating tests have been conducted. For example, the water solution of the aluminum compound is applied over the whole body, and a heat accumulation test has been conducted. From the result of the heat accumulation test, the following features have been proved. The main effect is not the antiperspirant effect but the water repellent effect. Further, a porous thin film, which easily transmits the sebum and the water, is formed. The invention has an effect of forming not only the degenerative stratum corneum but also a water-repellent film to a different solute in the water solution. The water-repellent film establishes a mechanism as if an interfacial phenomenon where the water is peeled from the solute occurs and this blocks the water. The following is considered. This mechanism acts as an effect to block the production of the emulsion, which will be the causative agent of itch. Alternatively, the mechanism demulsifies the emulsion and isolates the water and the sebum. Thus, the mechanism promotes the inclusion and absorption effects of the sebum with the cyclodextrin. The cyclodextrin that has absorbed and included the sebum forms free water and a colloidal emulsion. However, the concentration of water on the degenerative stratum corneum promotes a condensation effect and the cyclodextrin is gelatinized. This brings a favorable condition to absorb and secure the aqueous (Some of them are possibly oily.) causative agent of itch to between particles and the periphery of the CD. According to this, it is concluded that the itch is prevented. Thus, the present invention has been completed. Regarding this itch prevention effect, the itch prevention effect cannot be obtained in examples of single use of the respective aluminum chloride and cyclodextrin, which are described in detail in itch prevention effect experiment, which will be described later.

### BRIEF DESCRIPTION OF THE DRAWINGS

FIG. 1 is a photograph showing skin of a patient before applying the anti-itching agent of the present invention, with regard to Working Example 3.

FIG. 2 is a photograph showing skin of a patient before applying the anti-itching agent of the present invention, with regard to Working Example 3.

FIG. 3 is a photograph showing skin of a patient after applying the anti-itching agent of the present invention, with regard to Working Example 3.

FIG. 4 is a photograph showing skin of a patient after applying the anti-itching agent of the present invention, with regard to Working Example 3.

FIG. 5 is a photograph showing skin of a patient before applying the anti-itching agent of the present invention, with regard to Working Example 4.

FIG. 6 is a photograph showing skin of a patient after applying the anti-itching agent of the present invention, with regard to Working Example 4.

### DETAILED DESCRIPTION OF THE INVENTION

The following describes an aggressive factor related to an appearance of sudden itch not due to dermatitis and a mechanism of production of an aggressive material in detail. There are two main factors to cause sudden itching to appear, regardless of presence of an inflammation on a skin. The one factor is persistent sweat mainly secreted from apocrine sweat glands and eccrine sweat glands. Although the composition is mainly the water, the persistent sweat contains salt, even a trace of which becomes a stimulatory composition to the skin, uric acid, lactic acid, butyric acid, isovaleric acid, unsaturated aldehyde, and amines, which are produced by zymolysis of adhered bacteria and indigenous dermal bacteria. The other is sebum, which is unsaturated fatty acids secreted from sebaceous glands, and steroids. It is considered that when these aggressive factors are concentrated on stratum corneum and become a concentrated emulsion, the aggressive factors turn into the aggressive materials.

The mechanism of action is caused when the aggressive material meets the condition: (A) or (B) or (A)+(B).

(A) The aggressive material soaks into deep parts of epidermal cells and directly stimulates the Meissner's corpuscle, which is an end of a sensory nerve (C-fiber).

(B) When the aggressive material directly penetrates to the inside of the cells from the epidermal cell membrane, the cells themselves sense the aggressive material as an abnormal signal, and the result is recognized as itching.

When the water solution containing aluminum chloride, cyclodextrin and water as main ingredients is applied on a skin surface, the water solution instantly hardens the stratum corneum epidermidis. This constructs porous, degenerative stratum corneum epidermidis. The water solution of aluminum chloride and the cyclodextrin is entered in the porous stratum corneum epidermidis and a complex is formed. The emulsion, which is a cell disorder composition composed of sweat and sebum, is permeated into the stratum corneum, spread, concentrated by body temperature, transmitted through the stratum corneum and reached to the epidermal cells. In order to block the above state, the emulsion is accumulated by the complex and fixed. Accordingly, the concentrated emulsion cannot penetrate up to the epidermal cells.

If such a state is maintained, the skin surface of the stratum corneum can be always kept smooth. On a facial surface where the secretion of the sebum is particularly large, after an elapse of roughly 12 hours, an amount of absorption of the emulsion in the complex is likely to decay. Therefore, although a slightly sticky texture appears, even after an elapse of seven days, the itch does not occur. The reason is considered as follows. The formation of the complex barrier theoretically continues blocking the mechanism of production of the concentrated emulsion on the stratum corneum epidermidis until a turnover cycle of the epidermis. However, the entire skin surface constantly receives some sort of physical friction from, for example, clothes and a towel. Accordingly, around 36 hours is actually regarded as a limit for practical use.

When the accumulation of the emulsion by the complex barrier decays and is saturated, and an equilibrium state is reached, extra emulsion and newly produced sebum are oozed out to the stratum corneum surface without being included and absorbed. This phenomenon is caused by a surface-active effect generated on the interface of the stratum corneum. The phenomenon is considered to be caused by CD molecules filmy aligning with their hydrophobic faces (guest holes) downward and their hydrophilic groups upward. This generates an action and an effect of the hydrophilic group shedding the sebum and the sweat.

The application of the aluminum chloride solution and the cyclodextrin solution does not bring the action and the effect of inhibiting the sudden itch and the inflammation by themselves. However, the constitution of the emulsion barrier by the degenerative stratum corneum, the aluminum chloride, the cyclodextrin, and the water expresses the mechanism of action to effectively block the cycle of the dermatitis. Accordingly, the mechanism of action can also lead to a prevention of the itching caused by various kinds of inflammatory skin diseases.

The anti-itching agent of the present invention has an advantage that the anti-itching agent can be used even when a patient uses an ointment or the like for the therapy. In some cases, frequency of use of therapeutic medicine can be reduced and a period of treatment can be shortened. For a patient not desiring steroids for external application, adequate and satisfactory duration of use and new usage can be proposed. The following further describes problematic diseases with concrete examples in detail.

When the water solution in which the aluminum chloride and the cyclodextrin are added is applied over the whole body, the stickiness on the skin surface is reduced and a shine is reduced. When the water solution is applied over the entire facial surface after bathing and several hours are elapsed, the entire forehead is smooth and the rash skin touch disappears.

The reasons are considered as follows. The anti-itching agent of the present invention inhibits: 1) the expression of sudden itching and 2) penetration of the emulsion, which is constituted of the sweat and the sebum, related to an appearance of acute-phase folliculitis into the living epidermal cell layer and cells on a wall of hair follicle (a wall of the pores).

Besides, although there are countless various kinds of inflammatory skin diseases, the initial lesion is papular dermatitis. Apart from the seriousness of the dermatitis and whether the sudden itch is noticed or not, with the papular dermatitis, the sudden itch is preceded. After an elapse of certain time, the dermatitis develops into papules. To scratch these papules, the healthy skin around the papules, which is not directly related to the papules, is also scratched, resulting in expansion of the disease.

Further, the strength of scratch, whether the skin is strongly scratched by tiptoes, the skin is scratched so as to rub the skin by the pulps, the skin is scratched with a towel, or the skin is scratched over clothes; how long is the skin scratched and how long is the period of scratch; and under these situations, how long period and how strong extent the dermatitis keep occurring. These situations design and initialize various and variety of inflammatory skin diseases.

<Details of Itch Prevention Effect>
Purpose of Experiment: The water solution comprised of the aluminum chloride hydrate and the cyclodextrin (referred to as the CD) were each used alone for application to the skin over the whole body. From the result, whether the itch prevention effect was able to be bodily sensed or not was determined through somesthetic testing. The result was compared with the anti-itching agent of the present invention and listed as the experiment data 1, 2 and 3 below.
Participated Examinees: adult: three (male: 2, female: 1), infant: 1
Testing Method: The water solutions of Experiment 1 to Experiment 3 were applied to the skin over the whole body. The climbing of a low mountain (altitude: 300 to 400 m) of 2 to 20 km, running of 2 to 15 km, and an indoor test (infant: 1 person) were executed 100 times for all seasons and for one year with the respective samples. Whether the itch was clearly removed or not and the appearance of the itch was able to be prevented or not were measured setting eight hours from the test start as terminal time. During the period, the somesthetic testing was conducted for determination.
Determining Method: The total count of itches developed without application of the water solution to the skin over the whole body (the head, the face, the body, and the entire feet) was set to 100. The degree of the itch was calculated by an average value of three examinees and indicated by a “proportion of itch.”

<Experimental Example of Itch Prevention Effect>

Experiment 1.: The water solution containing 10% of aluminum chloride was applied over the whole body to determine whether the single use of the aluminum chloride was able to prevent the itch or not. The result is shown in the following.

<Result> The proportion of itch was 100%. The itch was not removed.

Experiment 2.: The somesthetic testing was conducted to determine whether the single use of the cyclodextrin was able to prevent the itch or not based on the following three prescription examples. The result is shown in the following.

1. The result of testing the application of the water solution containing 5% of α-CD to the skin over the whole body

<Result> The itch was not removed. The proportion of itch was 100%. Stickiness was felt on the skin as the skin became dried, and the itch was not removed.

2. The result of testing the application of the water solution containing 5% of β-CD to the skin over the whole body

<Result> Stickiness was felt on the skin. The proportion of itch was 100%. The itch was not removed.

3. The result of testing the application of the water solution containing 5% of γ-CD to the skin over the whole body

<Result> The itch was not removed. The proportion of itch was 100%. Stickiness was felt on the skin as the skin became dried, and the itch prevention effect was not able to be bodily sensed.

Experiment 3.: Based on <Working Example 1> of the present invention, 100 g of purified water, 8 g of aluminum chloride hexahydrate and 10 g of β-CD were entered in a flask and stirred and mixed well. Thus, colorless and transparent anti-itching agent was obtained. The pH of the obtained anti-itching agent was approximately 2.3, which was high value. However, many examinees didn't feel pricking stimulation, although one examinee felt stimulation because of the difference of the condition of the skin and personal difference.

<Result> The itch prevention effect could be felt. Stretched feeling and sticky feeling of the skin were not felt. The degree of the itch was 0 to 12% in all experiments. Thus, the itch prevention effect was bodily sensed. According to the Working example 1, 5 g of α-CD or γ-CD was used instead of the β-CD, and the water solution in which the aluminum chloride and one of the α-CD and the γ-CD were solved was applied to the skin for another test. No significant difference could be seen in the result.

<Consideration> In the experiments of 100 times, the itch prevention effect could be appropriately confirmed in all situations. Among them, 70% of the experiments felt that the itch was restricted enough to be bodily sensed, and 30% of the experiments could not feel the itch on the whole body at all. In other words, the proportion of itch was 5% or less.

To conduct the above-described experiment, it is considered that the state of the skin is not constant through the year and is affected by temperature/humidity conditions of the external air to some extent. However, even considering errors, the itch prevention effect has been verified from the experiment result of the anti-itching agent, although the experiment was the somesthetic testing.

The following describes a prescription example of the anti-itching agent of the present invention. The anti-itching agent containing 2 to 15 wt. % aluminum chloride hexahydrate or anhydrous aluminum chloride per 100 ml of water is a strong acid having a hydrogen ion density of approximately pH 1.8 to 2 regardless of the density. If the acidity is high, a patient feels strong stimulation on the skin. Therefore, the stimulation caused by a tiny wound can be significantly reduced if the acidity is adjusted to approximately pH 3.0 to 3.8 by reducing the density of the aluminum chloride or neutralizing the anti-itching agent with an alkaline agent if needed. If the acidity is high and an invisible wound is presented on the surface of the skin of a patient, the patient feels pricking stimulation as if the agent is penetrated into the wound. In order to reduce the stimulation, the acidity should be adjusted by neutralizing the anti-itching agent with an alkaline agent, such as sodium hydroxide. At this time, a reaction product such as sodium chloride and aluminum hydroxide is generated and the stimulation is increased by that. Therefore, if a flocculant such as a phosphate 2-starch, a xanthan gum and a polyacrylic acid is used, salts can be reduced to some extent. Of course, salts can be reduced by using conventionally known method.

As an example, the xanthan gum is used to reduce salts in the present invention. The salts, such as sodium chloride and aluminum hydroxide, generated when the aluminum chloride and the sodium hydroxide are reacted become a flocculated precipitate with the xanthan gum. Therefore, the salts can be reduced to some extent by filtering the flocculated precipitate. Alternatively, it is effective to use ion exchange resin for reducing salts.

When the anhydrous aluminum chloride dissolved in the purified water, there is a risk of a generation of hydrogen chloride gas and a spill caused by an exothermal reaction. This can be solved by adding other materials after the water solution is cooled.

The cyclodextrin (hereinafter referred to as CD) used in the present invention is an inclusion compound that can include a hydrophobic material, such as the sebum and the emulsion, by the interaction with the aluminum chloride. In the present invention, the cyclodextrin mainly means α-CD, β-CD, γ-CD, and the mixture composition of these materials. Furthermore, the derivative of α-CD, β-CD, and γ-CD, and inclusion compounds other than the CD can also be used. As the precondition, as long as the inclusion compound absorbs the emulsion containing the sebum and dissolves the emulsion into water, all inclusion compounds can be used. However, since the absorption condition changes depending on a solute to be added, the inclusion compound can be selected so as to establish an optimal condition. The available density of the cyclodextrin is 0.1 to 40 wt. % per 100 parts of purified water. In general, the itch can be suppressed when the density is within the range of 1 to 10%. The aluminum chloride is generally used within the range of 1 to 15%. In addition to the anhydrous aluminum chloride and the aluminum chloride hexahydrate, a polymer such as alum, basic aluminum chloride and chlorohydroxy aluminum can be used.

In particular, Alcl₃.6H₂O, which is a hydrate of the aluminum chloride, is efficient because it is relatively cheap and easily operated.

An antioxidant substance, which is also a usual moisturizer and a moisture composition, such as vitamin E and ascorbic acid 2-phosphate can be arbitrarily used. In addition, glycols such as propylene glycol, glycerin and 1,3-butylene glycol can be arbitrarily used as moisturizer. Furthermore, lower alcohols such as isopropyl alcohol and ethyl alcohol can be arbitrarily used as antibacterial agent, antiseptic agent and antimold agent.

If a thickener is added to the anti-itching agent, the anti-itching agent can be prevented from coming off from the coating surface and sustained release can be expected. Therefore, pasty and viscous anti-itching agent can be effective according to the purpose of use. For the thickener, water soluble thickeners such as mucopolysaccharide, dextrin, starch, xanthane gum, alginic acid, gum arabic, guar gum, mannan, kelco gel, gelatin, hydroxymethyl cellulose and hydroxyethyl cellulose; glycols such as polyethylene glycol, propylene glycol and glycerin; acrylic polymers such as polyacrylic acid; dextrins; and sugar alcohols can be used. However, the solution is agglutinated and gelated depending on the density of the aluminum chloride solution. Therefore, the thickener should be arbitrarily selected and used.

The following methods for applying the anti-itching agent, including jellied anti-itching agent, are easy and economical. The anti-itching agent is directly applied to the body with the pulps or the palm. Alternatively, usual methods such as spray type and sponge roll-on type are also easy and economical. In addition, the anti-itching agent can be mixed in detergents such as a shampoo and a body lotion.

The following describes the preferable embodiments of the present invention.

### Working Example 1

100 g of purified water, 12 g of aluminum chloride hexahydrate and 12 g of β-CD were entered in a flask, and then they were stirred and mixed well. Thus, colorless and transparent anti-itching agent was obtained. The pH of the obtained anti-itching agent was approximately 2.3, which was low value. Many examinees didn't feel pricking stimulation, although some examinees felt stimulation because of the difference of personal difference. As a supplementary explanation, when the anti-itching agent was sealed in a container and stored in a thermostatic chamber of 25° C., colonies of Aspergillus niger were identified. Therefore, it is considered that the aluminum chloride solution doses not have a bacteriostatic effect almost at all.

### Working Example 2

100 g of purified water, 10 g of aluminum chloride hexahydrate and 8 g of α-CD were entered in a flask, and then they were stirred and mixed well. Thus, colorless and transparent anti-itching agent was obtained. The pH of the obtained anti-itching agent was approximately 2.1, which was low value. Many examinees didn't feel pricking stimulation, although some examinees felt stimulation because of the difference of personal difference.

### Working Example 3

6.5 g of β-CD, 9 g (11 mL) of ethanol, 20 g of glycerin, 20 g of propylene glycol, 150 g of sodium hydroxide and 1.5 g of xanthane gum were added to 500 g of purified water, and then they were stirred and mixed to obtain water solution. Then, 44.5 g of anhydrous aluminum chloride was added and stirred. At last, the water solution was separated by a filter paper to separate sodium chloride, which was a reaction product, by coagulation and sedimentation. Thus, colorless and transparent anti-itching agent was obtained. In the experiment, the anhydrous aluminum chloride should be gradually added paying attention to a generation of gas and a spill. In addition, various materials should be added and mixed well after the exothermic reaction is completely finished.

### Concrete Example using Working Example 3

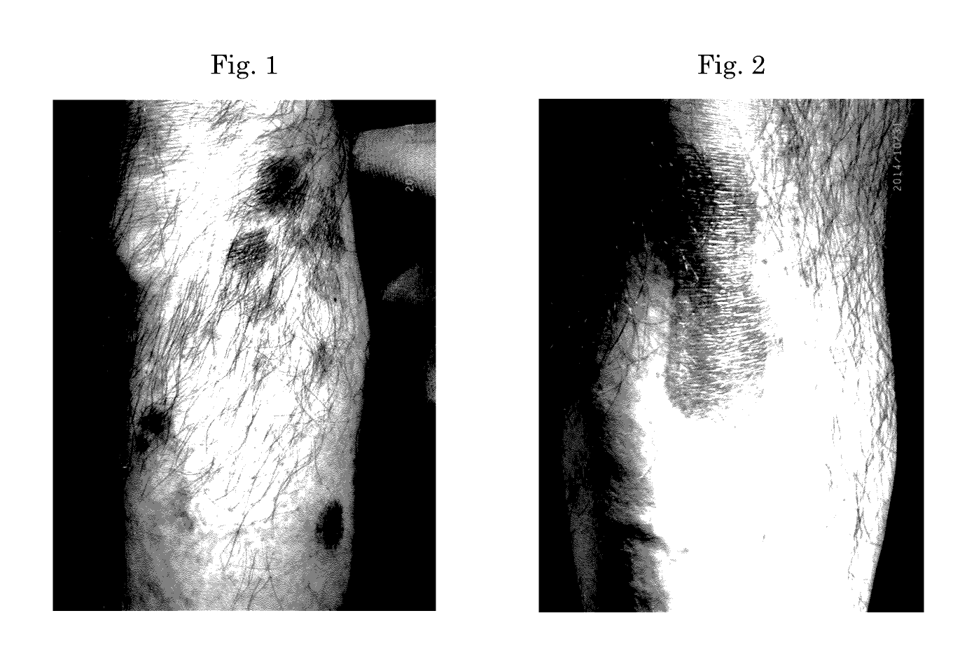

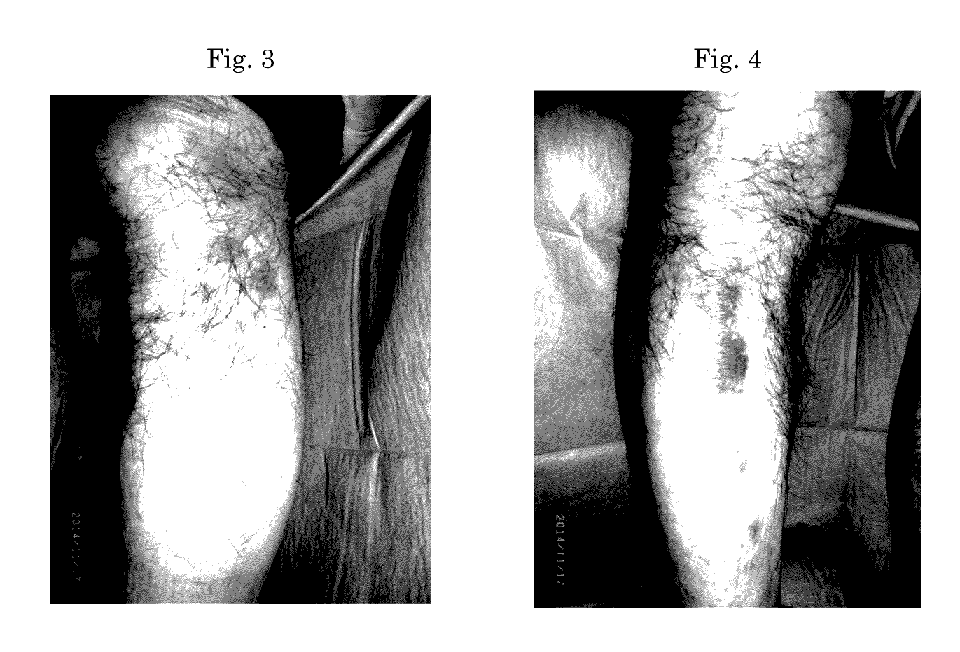

FIG. 1 and FIG. 2 show photographs of both lower limbs of a man in his seventies. The man suffered from refractory eczema of the lower limbs, the itch was not removed even when using the strongest class steroid for external application, the man scratched his lower limbs and the eczema was continued. When the working example 3 was firstly applied to the man, and then the external preparation containing steroid was used, the itch of the patient was removed and the eczema was completely cured in 17 days. FIG. 3 and FIG. 4 show the cured state.

### Working Example 4

3 g (1 g each) of α-CD, β-CD and γ-CD, 56 g of aluminum chloride hexahydrate, 9 g (11 mL) of ethanol, 20 g of glycerin, 20 g of propylene glycol, 150 g of sodium hydroxide and 2 g of xanthane gum were added to 500 g of purified water, and then they were stirred and mixed to obtain water solution. By using centrifugal separation, sodium chloride and aluminum hydroxide, which were a reaction product, were removed as a flocculated precipitate. Thus, colorless and transparent anti-itching agent was obtained.

### Concrete Example using Working Example 4

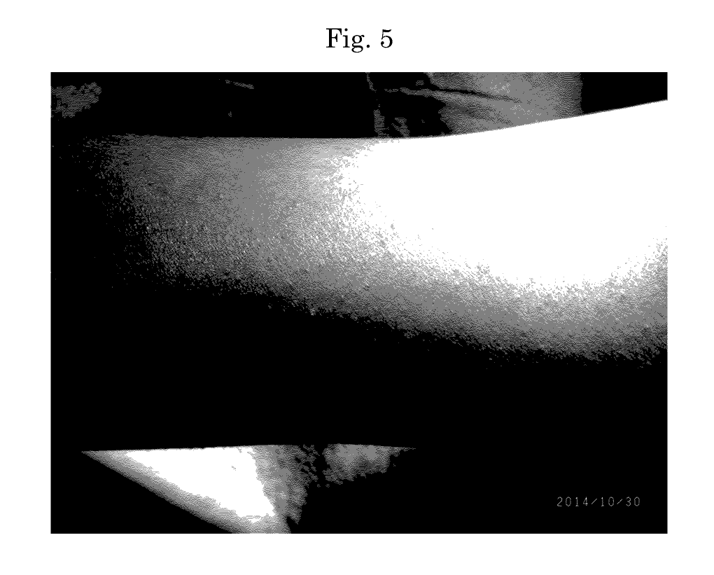

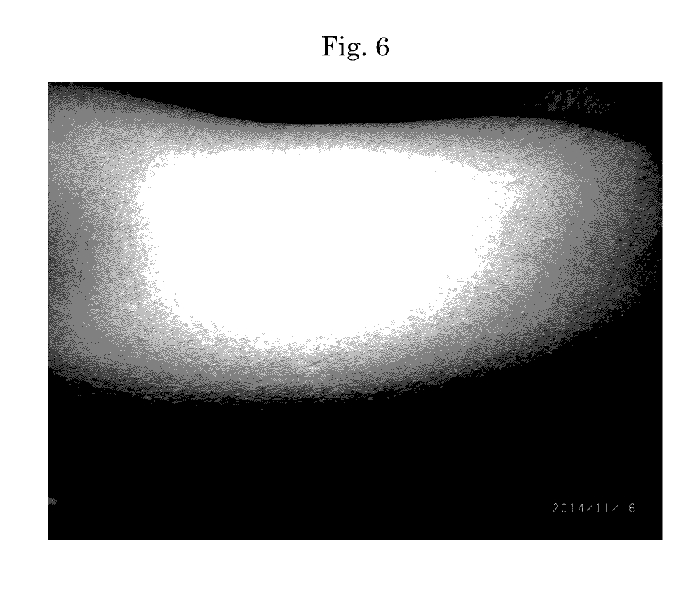

8-year old boy suffered from atopic dermatitis. When taking a bath, the boy scratched the whole body, especially both upper arms, because the itch was not removed. When the working example 4 was applied to the body every day before taking a bath. Before applying the working example 4, acute-phase folliculitis was seen a lot although they were not serious. FIG. 5 shows the photograph. After applying the working example 4, the eruption got better and the 8 year old boy said “there is no itch on the whole body.” FIG. 6 shows the photograph.

### Working Example 5

45.5 g of aluminum chloride hexahydrate, 6.5 g of β-CD, 20 g of glycerin, 20 g of propylene glycol, 9 g of 70% ethanol, 50 g of 10% sodium hydroxide were added to 500 g of purified water, and then they were stirred and mixed. At last, 1 g of xanthane gum was stirred and mixed, and the aluminum chloride and other flocculated precipitates were filtered. Thus, colorless and transparent anti-itching agent was obtained.

### Working Example 6

10 g of γ-CD, 60 g of chlorohydroxy aluminum, 10 g of anhydrous ethanol and 20 g of glycerin were added to 500 g of purified water, and then they were stirred and mixed to obtain water solution. Thus, colorless and transparent anti-itching agent was obtained.

The emulsion is the cell disorder composition, which becomes an ignition factor of acute inflammation or triggers the acute inflammation, or the emulsion causes a growth of bacteria. Inhibiting the production of the emulsion for a certain period of time (around 36 hours to two weeks) provides the following effects.

Except for the emulsion, as the factors for the pores to cause the inflammation, which is a part of a transition process to the dermatitis of the pores, influences from the ultraviolet rays, the steroid, and reactive oxygen have been known. However, the generation of the reactive oxygen means that the cell disorder has already been initialized from any cause.

The anti-itching agent inhibits a sequence of cycles of inflammation where the inflammation that has once occurred in the pores persists and then is reinforced little by little. It is said that the inflammation in the pores is increased and red eminences on the pores are caused by bacterial infection when touching and rubbing the pores. However, it is considered that the persistent penetration of the emulsion is largely related to the persistence and the aggravation of inflammation prior to secondary infection with bacteria. The process that the inflammation reaction persists and is reinforced is named as the ‘cycle of inflammation.’ The main factor bringing the cycle of inflammation is the emulsion.

The anti-itching agent has an effect of naturally regresses the red eminences, namely, the folliculitis, becoming larger on the pores. The anti-itching agent prevents the emulsion from keeping repeatedly stimulating the folliculitis. Thus, the anti-itching agent has an action and an effect that naturally flattens the red inflammatory eminences on the pores without developing the eminences into suppuration and an accumulation of pus.

Since strong itch, which happens suddenly, can be preliminarily prevented, the anti-itching agent reduces an occasion where patients with atopy and children sweat and scratch their heads, ears, external genitalias, and limb joints. This allows bringing effects not only preliminary preventing the appearance of eczema and dermatitis but also decreasing the range.

To validate the effects, it should be considered that there are various causes as the cause of the eczema and dermatitis except for the emulsion. The causes include, for example, the ultraviolet rays, medicines (a fomentation, an external medicine, and an oral medicine), chemical substances, insects, and plants.

The anti-itching agent has an effect of reducing keratosis pilaris (rashes on the cheek and the arm). Although the anti-itching agent does not cure this disease itself, an acute phase before the pores are cornified is the folliculitis (acute dermatitis of the pores). A trigger factor that initializes the inflammation of the folliculitis is an accumulation of the emulsions consisted of the sweat and the sebum on the stratum corneum epidermidis around the pores. The aluminum chloride adheres to the entire stratum corneum to prevent the accumulation of the water inside the stratum corneum. So to speak, this forms the sponge-like stratum corneum epidermidis into like a single plate.

The cornification of the keratosis pilaris means that the epidermal cells at the exit of the pores go through a denaturation and change into the inflammation, lose the nuclei, and are stacked, so-called accumulation of epidermis refuses. This forms the stratum corneum epidermidis at the exit of the pores into like the single plate to inhibit the accumulation of the emulsions. This allows preventing the inflammation and the denaturation of the cells. As a result, this prevents the cornification of the pores, and consequently ensuring preventing the keratosis pilaris. The effect can be felt in a short period (one to two weeks). This is because that the inflammation of the skin leading to the keratosis pilaris is much lighter than the inflammation of the acne.

The anti-itching agent has an action and effect of outstandingly preventing oxidized colors of the unsaturated fatty acid secreted from the sebaceous gland, mainly yellow stains on clothing generated by the secretion of the apocrine gland.

The stickiness of the sweat generated by excessive sweating on the hand is not only the water but also is the emulsion consisted of the sweat and the sebum. The surface-active effect, which is achieved by the interaction of the aluminum chloride and the CD, blocks and inhibits the emulsion accumulated on the stratum corneum epidermidis. This provides a smooth texture to a person who has touched the hand.

The anti-itching agent with the main ingredients of the aluminum chloride and the cyclodextrin is in a category of quasi-drug. Therefore, like an insect repellent, the anti-itching agent can be casually used, anytime and anywhere regardless of frequency. Accordingly, the anti-itching agent is also excellent in convenience.

The barrier complex for blocking the action of the emulsion is formed by the skin-degenerative stratum corneum, the aluminum chloride, the cyclodextrin and water. This reduces the folliculitis and the scratch action, which are causes of the itch. Therefore, various inflammatory skin diseases can be prevented. The anti-itching agent of the present invention has an advantage that can be used also during treatment with the ointment or a similar medicine. In some cases, this substantially reduces a usage frequency of curative medicine and duration of treatment. It is also possible to present an optimal, satisfactory periods of use or to propose a new usage method also for diseased patients who dislike steroids for external application. The anti-itching agent features extremely high effectiveness as one means for alternative medicine. The anti-itching agent is an invention that provides great anticipation and prospect for treatment strategy of these diseases.

Note that, this invention is not limited to the above-mentioned embodiments. Although it is to those skilled in the art, the following are disclosed as the one embodiment of this invention.
Mutually substitutable members, configurations, etc. disclosed in the embodiment can be used with their combination altered appropriately.
Although not disclosed in the embodiment, members, configurations, etc. that belong to the known technology and can be substituted with the members, the configurations, etc. disclosed in the embodiment can be appropriately substituted or are used by altering their combination.
Although not disclosed in the embodiment, members, configurations, etc. that those skilled in the art can consider as substitutions of the members, the configurations, etc. disclosed in the embodiment are substituted with the above mentioned appropriately or are used by altering its combination.

While the invention has been particularly shown and described with respect to preferred embodiments thereof, it should be understood by those skilled in the art that the foregoing and other changes in form and detail may be made therein without departing from the sprit and scope of the invention as defined in the appended claims.

What is claimed is:

1. Anti-itching agent, comprising:
a water solution containing an aluminum chloride, a purified water and a cyclodextrin as a main ingredient, wherein
a skin-degenerative stratum corneum is formed by applying the water solution on a skin,
a complex for blocking an emulsion is formed by the skin-degenerative stratum corneum, the aluminum chloride and the cyclodextrin, and
the emulsion produced by a persistent sweat and a sebum is blocked by the complex to prevent a sudden itch.

2. The anti-itching agent according to claim 1, wherein
the main ingredient contains 0.1 to 25 wt. % of the aluminum chloride per 100 parts of the purified water.

3. The anti-itching agent according to claim 1, wherein
the main ingredient contains 0.1 to 40 wt. % of the cyclodextrin per 100 parts of the purified water.

4. The anti-itching agent according to claim 1, wherein
the anti-itching agent is pasty and viscous.
